(19) United States
(12) Patent Application Publication
(10) Pub. No.: US 2011/0038880 A1
(43) Pub. Date: Feb. 17, 2011

(54) PARASPORIN-1 RECEPTOR AND USE THEREOF

(75) Inventors: Hideki Katayama, Munakata-shi (JP); Yoshitomo Kusaka, Kurume-shi (JP); Eiichi Mizuki, Kurume-shi (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: FUKUOKA PREFECTURAL GOVERNMENT, Fukuoka-shi (JP)

(21) Appl. No.: 12/935,513

(22) PCT Filed: Mar. 30, 2009
(86) PCT No.: PCT/JP2009/056548

§ 371 Date: Nov. 3, 2010

(30) Foreign Application Priority Data
Mar. 31, 2008 (JP) 2008-090190

(51) Int. Cl. A61K 39/395; A61K 38/16; A61P 35/00; G01N 33/567; C12N 5/02

(52) U.S. Cl. 424/174.1; 514/2.1; 424/130.1; 435/7.21; 435/375

(57) ABSTRACT

The receptor of parasporin-1 has been found to be beclin-1. Based on the finding, the present invention provides a method of determining the sensitivity of a cell to parasporin-1, comprising measuring the expression level of beclin-1 polypeptide on the cell membrane or in a membrane fraction of a cell; an inhibitor of the cytotoxicity of parasporin-1, containing beclin-1 polypeptide etc.; a method of screening for a substance capable of inhibiting the cytotoxicity of parasporin-1, comprising evaluating the degree of binding of parasporin-1 to beclin-1 polypeptide in the presence of a test substance; a cell death inducer containing an antibody that specifically recognizes beclin-1 polypeptide; a method of screening for a substance capable of inducing cell death, comprising evaluating the binding of a test substance to beclin-1 polypeptide, and the like.

### TECHNICAL FIELD

The present invention relates to a use of beclin-1 as a receptor of parasporin-1. More specifically, the present invention relates to a method and agent for determining the sensitivity of cells to parasporin-1 using beclin-1 or an antibody against beclin-1, a method and agent for inhibiting the cytotoxicity of parasporin-1, a method of screening for a cell death inducer, an antitumor agent or a substance capable of inducing cell death, and the like.

### BACKGROUND ART

Bacillus thuringiensis produces a proteinous inclusion body in the sporulation stage. This inclusion body is toxic to the larvae of certain insects (Non-patent Document 1). The present inventors discovered a toxin protein that is toxic to mammalian cells in large screening of inclusion bodies of B. thuringiensis (Non-patent Document 2). The biochemical properties of these proteins, including cell specificity and cytotoxicity, are very diverse; that is, some toxin proteins are toxic to a broad range of cell lines, and other proteins are toxic to a very limited range of cell lines (Non-patent Documents 2 to 5). These proteins differed from the Cyt protein of B. thuringiensis, which is toxic to mammalian cells and possesses hemolytic activity, because these proteins did not exhibit hemolytic activity.

Parasporin proteins can roughly be classified into four groups according to primary structure similarity, and 13 kinds of parasporin proteins have been identified so far (see the website http://parasporin.fitc.pref.fukuoka.jp/). The individual parasporin proteins have totally different specificities to cells and molecular weights.

Parasporin-1 is produced as an 81-kDa toxin precursor by the B. thuringiensis A1190 strain, and exhibits cytotoxicity upon partial degradation by treatment with a protease such as trypsin. The active form parasporin-1 is a complex consisting of a 56-kDa protein and a 15-kDa protein, exhibiting specific cytotoxic activity (Non-patent Document 14). HeLa, which is a human cervical carcinoma cell line, and HL-60, which is a human promyelocytic leukemia cell line, are highly sensitive to parasporin-1, whereas normal human uterine smooth muscle cells and Caco-2, which is a colorectal cancer cell line, are little sensitive. So far, in a test of the sensitivity of 15 kinds of cell lines, including normal cell lines, to parasporin, sensitivity was noted in 4 cell lines. This demonstrates that parasporin-1 is a cytotoxin possessing extremely high cell specificity (Non-patent Document 14). Parasporin-1, in its toxicity induction process, induces an elevation of the concentration of cytoplasmic free Ca²⁺ accompanying a Ca²⁺ influx, which triggers cell death. Since this was suppressed by suramin, which is an inhibitor of G-protein, it has been estimated that the Ca²⁺ influx induced by parasporin-1 is mediated by an intracellular signal transduction system via G-protein (Non-patent Document 6). Furthermore, in parasporin-1-treated HeLa cells, not only degradation of Poly(ADP-ribose) polymerase and Caspase-3 activation (Non-patent Document 6), but also degradation of DNA into nucleosome units and translocation of phosphatidylserine are noted; therefore, parasporin-1 is a toxin that induces apoptosis to target cells.

Because parasporin-1 is toxic to a very limited range of cells and induces apoptosis to targeted cells, its applications to the fields of pharmaceuticals, cell identifying reagents, and cell biology are expectable by understanding the action mechanisms of parasporin-1. Particularly, because the binding of the toxin to a receptor is the most fundamental reaction in the toxic action, its identification is thought to be important. However, the receptor of parasporin-1 has not yet been identified.

Meanwhile, beclin-1 is a publicly known protein first identified as an autophagy-related protein (Non-patent Document 7), and has been reported to act as a cancer suppressor (Non-patent Documents 8 to 10). Furthermore, beclin-1 has been reported to be an intracellular protein that interacts with Bcl-2 and Bcl-X_{L}, which are factors that control apoptosis (Non-patent Documents 11 to 13). However, it is not known at all that beclin-1 protein is capable of being exposed to cell surfaces, and that beclin-1 is capable of functioning as a receptor.
non-patent document 1: Schnepf, E. et al., Microbiol. Mol. Biol. Rev. 62, 775-806 (1998)
non-patent document 2: Mizuki, E. et al., J. Appl. Microbiol. 86, 477-486 (1999)
non-patent document 3: Ito, A. et al., J. Biol. Chem. 279, 21282-21286 (2004)
non-patent document 4: Lee, D. W. et al., Biochem. Biophys. Res. Commun. 272, 218-223 (2000)
non-patent document 5: Yamashita, S. et al., Can J. Microbiol. 46, 913-919 (2000)
non-patent document 6: Katayama, H. et al., J. Biol. Chem. 282, 7742-7752 (2007)
non-patent document 7: Liang, H. X. et al., Nature 402, 672-676 (1999)
non-patent document 8: Atia, V. M. et al., Genomics 59, 59-65 (1999)
non-patent document 9: Qu, X. et al., J. Clin. Investig. 112, 1809-1820 (2003)
non-patent document 10: Yue, Z. et al., Proc. Natl. Acad. U.S. A., b, 15077-15082(2003)
non-patent document 11: Pattingre, S. at al., Cell, 122, 927-939 (2005)
non-patent document 12: Liang, X. H. et al., J. Virol. 72, 8586-8596 (1998)
non-patent document 13: Furuya, D. et al., Exp. Cell Res. 307, 26-40 (2005)
non-patent document 14: Katayama, H. et al., J. Biochem. 137, 17-25 (2005)

### DISCLOSURE OF THE INVENTION

### Problems to be Solved by the Invention

In view of the above-described circumstances, the present invention aims to identify the parasporin-1 receptor on a target cell, which is thought to be most important in the induction of the cytotoxicity of parasporin-1, and provide a new antitumor drug that targets the receptor, a screening method therefor, a tool for cell sorting and the like.

### Means of Solving the Problems

The present inventors, in order to explain the cell specificity of parasporin-1, searched for, and identified, the receptor of parasporin-1 using a photo-reactive chemical cross linker. Several candidate proteins for the receptor of parasporin-1 were separated by two-dimensional electrophoresis, and analyzed by the mass finger printing method utilizing a mass spectrometer; as a result, it was found that the parasporin-1-binding protein is beclin-1. An analysis using an anti-beclin-1 antibody and flow cytometry showed that beclin-1, which had been thought to be an intracellular protein, was localized outside of the cell membrane, and the binding of parasporin-1 to HeLa cells was inhibited by the anti-beclin-1 antibody. Furthermore, when the expression level of beclin-1 exposed on cell surfaces by the anti-beclin-1 antibody was examined, the expression level was high in HeLa cells, which are parasporin-1-sensitive cells, and the expression level was extremely low in Caco-2 and normal uterine smooth muscle cells, which are non-parasporin-1-sensitive cells. The anti-beclin-1 antibody (anti-peptide antibody) suppressed the binding of parasporin-1 to cell surfaces, and inhibited the cytotoxic effect of parasporin-1. Meanwhile, the anti-beclin-1 antibody (as a whole) exhibited cytotoxic activity by itself, suggesting that in the cell death, caspase-8 activation may be involved as with parasporin-1. In the results above, the present inventors found that the receptor of parasporin-1 is beclin-1, and that beclin-1 exposed on cell surfaces is one factor to determine the parasporin-1 sensitivity of cells, and have completed the present invention described below.

Accordingly, the present invention related to the following:
[1] A method of determining the sensitivity of a cell to parasporin-1, comprising measuring the expression level of beclin-1 polypeptide on the cell membrane or in a membrane fraction of a cell, and determining the sensitivity of the cell to parasporin-1 on the basis of a positive correlation between the expression level of beclin-1 polypeptide and the sensitivity to parasporin-1.
[2] The method described in [1], wherein the measurement of the expression level of beclin-1 polypeptide is performed using an antibody that specifically recognizes beclin-1 polypeptide.
[3] An agent for determining the sensitivity of a cell to parasporin-1, comprising an antibody that specifically recognizes beclin-1 polypeptide.
[4] An inhibitor of the cytotoxicity of parasporin-1, comprising beclin-1 polypeptide or an antibody that specifically recognizes beclin-1 polypeptide.
[5] A method of inhibiting the cytotoxicity of parasporin-1, comprising administering beclin-1 polypeptide or an antibody that specifically recognizes beclin-1 polypeptide.
[6] A method of screening for a substance capable of inhibiting the cytotoxicity of parasporin-1, comprising the steps shown below:
(I) evaluating the degree of binding of parasporin-1 to beclin-1 polypeptide in the presence of a test substance, and
(II) selecting a substance that has inhibited the binding of parasporin-1 to beclin-1 polypeptide as a substance capable of inhibiting the cytotoxicity of parasporin-1.
[7] A combination comprising parasporin-1 and beclin-1 polypeptide.
[8] A cell death inducer comprising an antibody that specifically recognizes beclin-1 polypeptide.
[9] An antitumor agent comprising an antibody that specifically recognizes beclin-1 polypeptide.
[10] A method of inducing cell death, comprising bringing an antibody that specifically recognizes beclin-1 polypeptide into contact with a cell.
[11] The method described in [10], wherein the cell is a tumor cell.
[12] A method of screening for a substance capable of inducing cell death, comprising the steps shown below:
(I) evaluating the degree of binding of a test substance to beclin-1 polypeptide, and
(II) selecting a test substance that has bound to beclin-1 polypeptide as a substance capable of inducing cell death.
[13] The method described in [12], wherein the beclin-1 polypeptide used in (I) is beclin-1 polypeptide expressed on the cell membrane.
[14] An antibody that specifically recognizes beclin-1 polypeptide, for use in inducing cell death.
[15] An antibody that specifically recognizes beclin-1 polypeptide, for use in preventing or treating a tumor.
[16] A method of inducing cell death in a mammal, comprising administering to the mammal an effective amount of an antibody that specifically recognizes beclin-1 polypeptide.
[17] A method of preventing or treating a tumor in a mammal, comprising administering to the mammal an effective amount of an antibody that specifically recognizes beclin-1 polypeptide.

### Effect of the Invention

Provided by the present invention are various uses of beclin-1 as a receptor of parasporin-1.

Using the method of the present invention for determining sensitivity to parasporin-1, it is possible to objectively determine the sensitivity of various cells to parasporin-1 on the basis of the expression level of parasporin-1 receptor definitely supported by molecular biologically.

Using the inhibitor of the cytotoxicity of parasporin-1 of the present invention, it is possible to potently suppress the cell death (apoptosis) induced by parasporin-1.

Using the cell death inducer of the present invention, it is possible to induce cell death (apoptosis) specifically to cells expressing beclin-1 polypeptide on the cell membrane or in a membrane fraction, and the inducer is useful as a cell-specific antitumor agent.

### BRIEF DESCRIPTION OF THE DRAWINGS

FIG. 1 shows the binding of parasporin-1 labeled with Alexa Fluor 488 dye to HeLa cells. (A) Observations using a light microscope (A-C) and a fluorescence microscope (D-F). (B) Observations using a flow cytometer.

FIG. 2 shows correlations between parasporin-1 sensitivity and parasporin-1 affinity. (A) Parasporin-1 susceptibilities of various cell lines. (B) Affinity of parasporin-1 to cell surfaces of various cell lines.

FIG. 3 shows screening for and identification of parasporin-1 receptor using SBED-labeled parasporin-1. (A) Detection of parasporin-1-binding protein by Western blotting (one-dimensional electrophoresis). (B and C) Separation of parasporin-1-binding protein by two-dimensional electrophoresis. Left panel: control (SBED-labeled BSA). Right panel: SBED-labeled parasporin-1.

FIG. 4 shows effects of anti-beclin-1 antibody (upper panel), anti-TMOD-3 antibody (middle panel) and anti-cytochrome C antibody (lower panel) on the binding of parasporin-1 to HeLa cells.

FIG. 5 shows the localization of beclin-1 to the cell membrane. (A) Flow cytometric analysis of beclin-1 expression on HeLa cell surface. (B) Analysis of beclin-1 expression by Western blotting. Upper panel: whole cells, lower panel: membrane fractions containing organelle.

FIG. 6 shows cell death of HeLa cells induced by anti-beclin-1 antibody.

### BEST MODE FOR EMBODYING THE INVENTION

### 1. Method and Agent for Determining the Sensitivity of Cells to Parasporin-1

The present invention provides a method of determining the sensitivity of cells to parasporin-1. The method of sensitivity determination of the present invention comprises measuring the expression level of beclin-1 polypeptide on the cell membrane or in a membrane fraction of a cell, and determining the sensitivity of the cell to parasporin-1 on the basis of a positive correlation between the expression level and sensitivity to parasporin-1. The method of sensitivity determination of the present invention is useful for, for example, distinguishing tumors on which an antitumor drug is effective and tumors on which the same is ineffective, in developing parasporin-1 as the antitumor drug.

Parasporin-1 is a publicly known protein that was isolated from the B. thuringiensis A1190 strain (Katayama, H. et al., J. Biochem. 137, 17-25 (2005)). Parasporin-1 is produced as an about 81-kDa precursor, and upon partial degradation by protease treatment, it forms a complex consisting of an about 56-kDa polypeptide and an about 15-kDa polypeptide. This complex possesses binding activity for beclin-1, and binds to beclin-1 expressed on a cell surface to induce cell death due to apoptosis to the cell. Herein, “parasporin-1” means this active complex unless otherwise stated.

“The sensitivity of a cell to parasporin-1” refers to the degree of inducibility of cell death (particularly apoptosis) when the cell is exposed to parasporin-1. “A cell is sensitive to parasporin-1” means that cell death (particularly apoptosis) is induced when the cell is exposed to parasporin-1.

Cells used in the method of sensitivity determination of the present invention are mammalian cells. Mammals include laboratory animals, including rodents such as mice, rats, hamsters and guinea pigs, and rabbits; domestic animals such as pigs, cattle, goat, horses, sheep, and minks; companion animals such as dogs and cats; primates such as humans, monkeys, rhesus monkeys, marmosets, orangutans, and chimpanzees, and the like. Cells used in the method of sensitivity determination of the present invention are preferably human cells.

Cells used in the method of sensitivity determination of the present invention include cultured cells, cells separated from living organisms, cells comprised in tissues separated from living organisms, cells in living organisms and the like. Cells used in the method of sensitivity determination of the present invention are tumor cells or non-tumor cells, preferably tumor cells.

Beclin-1 is a publicly known protein (Liang, H. X. et al., Nature 402, 672-676 (1999); Atia, V. M. et al., Genomics 59, 59-65 (1999); Qu, X. et al., J. Clin. Investig. 112, 1809-1820 (2003); Yue, Z. et al., Proc. Natl. Acad. Sci. U.S.A., b, 15077-15082 (2003); Pattingre, S. et al., Cell, 122, 927-939 (2005); Liang, X. H. et al., J. Virol. 72, 8586-8596 (1998); Furuya, D. et al., Exp. Cell Res. 307, 26-40 (2005)), and the amino acid sequence and mRNA (cDNA) sequence thereof are also publicly known. The beclin-1 that can be used in the present invention is the beclin-1 of one of the above-described mammals. As the amino acid sequence of human beclin-1, the amino acid sequence shown by SEQ ID NO:2 is known (NCBI Integrated Database Accession No. NP_{—}003757, version NP_{—}003757.1); as the nucleotide sequence of human beclin-1 mRNA (cDNA) that encodes the same, the nucleotide sequence shown by SEQ ID NO:1 is known (NCBI Integrated Database Accession No. NM_{—}003766, version NM_{—}003766.2/coding region: 134-1486). As orthologs thereof, mouse beclin-1 [amino acid sequence: SEQ ID NO:4 (NCBI Integrated Database Accession No. NP_{—}062530, version NP_{—}062530.2), mRNA(cDNA) nucleotide sequence: SEQ ID NO:3 (NCBI Integrated Database Accession No. NM_{—}019584, version NM_{—}019584.3/coding region: 139-1485)], rat beclin-1[amino acid sequence: SEQ ID NO:6 (NCBI Integrated Database Accession No. NP_{—}446191, version NP_{—}446191.1), mRNA (cDNA) nucleotide sequence: SEQ ID NO:5 (NCBI Integrated Database Accession No. NM_{—}053739, version NM_{—}053739.2/coding region: 225-1571)] and the like are known.

A measurement of the expression level of beclin-1 polypeptide on the cell membrane or in a membrane fraction is normally performed ex vivo.

The expression level of beclin-1 polypeptide can be measured by an immunochemical technique using an antibody that specifically recognizes beclin-1 polypeptide. Immunochemical techniques include flow cytometric analysis, radioimmunoassay (RIA method), ELISA (Methods in Enzymol. 70: 419-439 (1980)), Western blotting, immunohistological staining and the like.

Parasporin-1 binds to beclin-1 expressed on the cell surface and induces cell death to the cell. Therefore, the expression level of beclin-1 polypeptide on the cell membrane (that is, on the cell surface) or in a membrane fraction better correlates with the sensitivity of the cell to parasporin-1 than the expression level of beclin-1 polypeptide in the whole cell. Therefore, in the method of sensitivity determination of the present invention, the expression level of beclin-1 polypeptide on the cell membrane or in a membrane fraction, more preferably the expression level of beclin-1 polypeptide on the cell membrane, is measured. “A membrane fraction” includes the membranes constituting organellae and the cell membrane. A membrane fraction can be prepared from a cell by a method known per se such as centrifugation. In measuring the expression level of beclin-1 polypeptide on the cell membrane, immunochemical techniques such as flow cytometric analysis and immunohistological staining are suitably used. In measuring the expression level of beclin-1 polypeptide in a membrane fraction, immunochemical techniques such as radioimmunoassay (RIA method), ELISA (Methods in Enzymol. 70: 419-439 (1980)), and Western blotting are suitably used.

An antibody that specifically recognizes beclin-1 polypeptide can be produced by conventional method of antibody production using beclin-1 polypeptide or a partial peptide having the antigenicity thereof as an immunogen. As mentioned herein, antibodies include, but are not limited to, natural type antibodies such as polyclonal antibodies and monoclonal antibodies (mAbs), chimeric antibodies, humanized antibodies, single-stranded antibodies and human antibodies that can be produced using gene recombination technology, and binding fragments thereof. Preferably, the antibody is a polyclonal antibody, a monoclonal antibody or a binding fragment thereof. A binding fragment means a partial region of one of the above-described antibodies having the specific binding activity; specifically, for example, F(ab′)₂, Fab′, Fab, Fv, sFv, dsFv, sdAb and the like can be mentioned (Exp. Opin. Ther. Patents, Vol. 6, No. 5, p. 441-456, 1996). The class of antibody is not particularly limited; antibodies of any itotypes such as IgG, IgM, IgA, IgD and IgE are encompassed. Preferably, the class is IgG or IgM, and in view of the ease of purification and the like, the class is more preferably IgG.

Next, on the basis of the measured expression level of beclin-1 polypeptide on the cell membrane or in a membrane fraction, the sensitivity of a cell to parasporin-1 is determined. As shown in an Example described below, as the expression level of beclin-1 polypeptide on the cell membrane or in a membrane fraction in a cell increases, the sensitivity of the cell to parasporin increases. The above-described determination is made on the basis of such a positive correlation between the expression level of beclin-1 polypeptide and sensitivity to parasporin-1.

In an embodiment, for example, if the cell expresses beclin-1 polypeptide on the cell membrane or in a membrane fraction, it can be determined that the cell is sensitive to parasporin-1. If the cell does not express beclin-1 polypeptide on the cell membrane or in a membrane fraction, it can be determined that the cell is non-sensitive to parasporin-1.

In another embodiment, for example, a cell that does not express beclin-1 polypeptide on the cell membrane or in a membrane fraction, and that is non-sensitive to parasporin-1 (negative control), and a cell that expresses beclin-1 polypeptide on the cell membrane or in a membrane fraction, and that is sensitive to parasporin-1 (positive control), are provided in advance, and the expression level of beclin-1 polypeptide on the cell membrane or in a membrane fraction in the measurement subject cell is compared with those of the positive control and negative control. Alternatively, a diagram of the correlation between the expression level of beclin-1 polypeptide on the cell membrane or in a membrane fraction in a cell and the sensitivity to parasporin-1 may be generated in advance, and the expression level of beclin-1 polypeptide on the cell membrane or in a membrane fraction in the measurement subject cell may be compared with the correlation diagram. This comparison of expression level is preferably made on the basis of the presence or absence of a significant difference.

Then, judging from the comparative results for beclin-1 polypeptide expression levels, if the beclin-1 polypeptide expression level on the cell membrane or in a membrane fraction in the measurement subject cell is relatively high, it can be determined that the sensitivity of the cell to parasporin-1 is relatively high. Conversely, if the beclin-1 polypeptide expression level on the cell membrane or in a membrane fraction in the measurement subject cell is relatively low, it can be determined that the sensitivity of the cell to parasporin-1 is relatively low.

The present invention also provides an agent for determining the sensitivity of a cell to parasporin-1, containing the above-described antibody that specifically recognizes beclin-1 polypeptide (referred to as the agent (1) of the present invention). The agent (I) of the present invention can be a kit for determining the sensitivity of a cell to parasporin-1. By using the agent (I) of the present invention, it is possible to easily determine the sensitivity of a cell to parasporin-1 by the above-described method.

An antibody that specifically recognizes beclin-1 polypeptide can be dissolved in water or an appropriate buffer solution (e.g., TE buffer, PBS and the like) to obtain an appropriate concentration, and preserved at about −20° C. to 4° C.

The agent (I) of the present invention may further contain other ingredients necessary for embodying the method according to the method of measuring the expression level of beclin-1 polypeptide.

For example, the agent (I) of the present invention can further contain a labeled secondary antibody, color developing substrate, blocking liquid, washing buffer solution, ELISA plate, blotting membrane and the like.

### 2. Agent and Method for Inhibiting the Cytotoxicity of Parasporin-1

As stated above, parasporin-1 binds to beclin-1 polypeptide on the cell surface of a target cell to induce cell death (particularly apoptosis) to the cell. Therefore, by administering an effective amount of beclin-1 polypeptide or an antibody that specifically recognizes beclin-1 polypeptide, the binding of parasporin-1 to beclin-1 polypeptide on the cell surface is inhibited, and the cytotoxicity of parasporin-1 is inhibited. Therefore, the present invention provides an inhibitor of the cytotoxicity of parasporin-1 comprising beclin-1 polypeptide or an antibody that specifically recognizes beclin-1 polypeptide (the inhibitor of the present invention).

The beclin-1 polypeptide that can be used in the present invention is the beclin-1 polypeptide of one of the above-described mammals. As mammalian beclin-1 polypeptides, human beclin-1 polypeptides (for example, a polypeptide comprising the amino acid sequence shown by SEQ ID NO:2), mouse beclin-1 polypeptides (for example, a polypeptide comprising the amino acid sequence shown by SEQ ID NO:4), rat beclin-1 polypeptides (for example, a polypeptide comprising the amino acid sequence shown by SEQ ID NO:6) and the like can be mentioned. Beclin-1 polypeptide can be obtained by culturing a transformant incorporating a beclin-1 expression vector designed on the basis of the gene sequence thereof, and isolating/purifying the desired product from the culture using a commonly known means of purification such as column chromatography.

As an antibody that specifically recognizes beclin-1 polypeptide, an antibody that can be used in the above-described method of sensitivity determination of the present invention can be used; as the antibody, an antibody capable of inhibiting the specific binding of beclin-1 polypeptide and parasporin-1 polypeptide (neutralizing antibody) is preferable. The antibody used for the inhibitor of the present invention is preferably an antibody that does not exhibit an activity to induce cell death to a beclin-1-expressing cell when brought into contact with the cell. The epitope recognized by the antibody used in the inhibitor of the present invention is not particularly limited, as far as the antibody inhibits the cytotoxicity of parasporin-1. As an excellent antibody that potently inhibits the cytotoxicity of parasporin-1 and little induces cell death to beclin-1-expressing cells, an antibody that recognizes a site of human beclin-1 polypeptide comprising threonine 72 (Thr72) as an epitope can be mentioned.

The inhibitor of the present invention may contain, in addition to the above-described beclin-1 polypeptide or antibody that specifically recognizes beclin-1 polypeptide, an optionally chosen carrier, for example, a pharmaceutically acceptable carrier.

As examples of the pharmaceutically acceptable carrier, excipients such as sucrose and starch; binders such as cellulose and methylcellulose; disintegrants such as starch and carboxymethylcellulose; lubricants such as magnesium stearate and Aerosil; flavoring agents such as citric acid and menthol; preservatives such as sodium benzoate and sodium hydrogen sulfite; stabilizers such as citric acid and sodium citrate; suspending agents such as methylcellulose and polyvinylpyrrolidone; dispersing agents such as surfactants; diluents such as water and physiological saline; base waxes; and the like can be mentioned, which, however, are not to be construed as limiting.

The content of beclin-1 polypeptide or an antibody that specifically recognizes beclin-1 polypeptide in the inhibitor of the present invention is, for example, about 0.01 to 100% is by weight of the entire pharmaceutical composition.

In an embodiment, the inhibitor of the present invention is used in vivo. In this case, the inhibitor is orally or parenterally administered to a subject animal. As preparations suitable for oral administration, liquids, capsules, sachets, tablets, suspensions, emulsions and the like can be mentioned. Preparations suitable for parenteral administration (for example, subcutaneous injection, intramuscular injection, topical injection, intraperitoneal administration and the like) include aqueous and non-aqueous isotonic sterile injectable liquids, which may comprise an antioxidant, a buffer solution, a bacteriostatic agent, an isotonizing agent and the like. Aqueous and non-aqueous sterile suspensions can also be mentioned, which may comprise a suspending agent solubilizer, a thickening agent, a stabilizer, an antiseptic and the like. These preparations can be encapsulated in containers such as ampoules and vials for unit dosage or a plurality of dosages. It is also possible to freeze-dry the active ingredient and a pharmaceutically acceptable carrier, and store the preparation in a state that may be dissolved or suspended in an appropriate sterile vehicle just before use.

When the inhibitor of the present invention is used in vivo, the dosage of the inhibitor of the present invention varies depending on the activity and choice of the active ingredient, seriousness of illness, recipient animal species, the recipient's drug receptivity, body weight, age, and the like, and cannot be generalized; however, the dosage is normally about 0.001 to about 500 mg/kg, based on the amount of active ingredient, per day for an adult human.

The inhibitor of the present invention is safely administered to the above-described mammal in a way such that the active ingredient thereof will be delivered to a cell that is a target of parasporin-1 (a cell that expresses beclin-1 on cell surface).

In another embodiment, the inhibitor of the present invention is used in vitro. In this case, beclin-1 polypeptide or an antibody that specifically recognizes beclin-1 polypeptide is administered (added) to a medium comprising parasporin-1 and a target cell therefor (a cell that expresses beclin-1 on cell surface).

When the inhibitor of the present invention is used in vitro, the dosage of the inhibitor of the present invention varies depending on the activity and choice of the active ingredient and the like, and cannot be generalized; however, the dosage is normally about 0.1 to 500 μg/ml, based on the final concentration of active ingredient in the culture.

Because beclin-1 polypeptide or an antibody that specifically recognizes beclin-1 polypeptide is capable of inhibiting the binding of parasporin-1 to beclin-1 polypeptide on cell surface to inhibit the manifestation of cytotoxicity by parasporin-1, the inhibitor of the present invention is useful in preventing/treating cell death and poisoning that can be induced by parasporin-1. The inhibitor of the present invention is also useful in analyzing the mechanism for the manifestation of cytotoxicity by parasporin-1.

### 3. Method of Screening for Substances Capable of Inhibiting the Cytotoxicity of Parasporin-1

The present invention provides a method of screening for substances capable of inhibiting the cytotoxicity of parasporin-1, comprising the steps shown below (the screening method (1) of the present invention):
(I) evaluating the degree of binding of parasporin-1 to beclin-1 polypeptide in the presence of a test substance, and
(II) selecting a substance that has inhibited the binding of parasporin-1 to beclin-1 polypeptide as a substance capable of inhibiting the cytotoxicity of parasporin-1.

The step (I) can further comprise comparing the degree of binding of parasporin-1 to beclin-1 polypeptide in the presence of the test substance with the degree of binding of parasporin-1 to beclin-1 polypeptide in the absence of the test substance.

The test substance subjected to the screening method (I) of the present invention may be any commonly known compound or a novel compound; examples include nucleic acids, sugars, lipids, proteins, peptides, organic low molecular compounds, compound libraries prepared using combinatorial chemistry technology, random peptide libraries, or naturally occurring ingredients derived from microorganisms, animals, plants, marine organisms and the like, and the like.

As stated above, parasporin-1 is produced as an about 81-kDa precursor, and upon partial degradation by protease treatment, it forms an active (possessing cytotoxicity) complex consisting of an about 56-kDa polypeptide and an about 15-kDa polypeptide. In the screening method (I) of the present invention, this active complex is preferably used as parasporin-1. The parasporin-1 used in the screening method (I) of the present invention is preferably one that has been isolated and purified. “Isolated and purified” means having been treated to remove compounds other than the desired product.

In the screening method (I) of the present invention, a beclin-1 polypeptide that can be used in the above-described inhibitor of the present invention can be used as beclin-1 polypeptide. In an embodiment, the beclin-1 polypeptide used in the screening method (I) of the present invention has been isolated and purified. In another embodiment, beclin-1 polypeptide expressed on the cell surface of a cell naturally or forcibly expressing beclin-1 (preferably a cell of one of the above-described mammals) is used in the screening method (I) of the present invention. The cell naturally expressing beclin-1 is not particularly limited, as far as it potentially expresses beclin-1; as the cell, a primary culture cell, a cell line induced from the primary culture cell and the like can be used. Examples of cells naturally expressing beclin-1 include cervical carcinoma cells (HeLa cells and the like). Cells forcibly expressing beclin-1 include a transformant incorporating an expression vector capable of expressing beclin-1, and the like. In the vector, a polynucleotide that encodes beclin-1 polypeptide is integrated downstream of a promoter functional in the host cell in a way such that beclin-1 can be expressed in the host cell.

The evaluation in the step (I) is normally performed in an appropriate culture medium or buffer solution. The degree of binding of parasporin-1 to beclin-1 polypeptide can be evaluated by a method of interaction analysis well known in the art, for example, surface plasmon resonance, binding assay, immunological techniques and the like.

For example, parasporin-1 and beclin-1 polypeptide are brought into contact with each other in the presence of a test substance by immobilizing either parasporin-1 or beclin-1 polypeptide on a chip, and loading a solution containing the other polypeptide and the test substance on the chip. Next, the binding and dissociation of parasporin-1 to and from beclin-1 polypeptide are measured in the presence of the test substance by the surface plasmon resonance method, and are compared with the binding and dissociation obtained when a control solution without the test substance is loaded on the chip.

Alternatively, parasporin-1 and beclin-1 polypeptide are brought into contact with each other in the presence of a test substance by immobilizing either parasporin-1 or beclin-1 polypeptide on a plate, and adding a solution containing the other polypeptide labeled with an appropriate radioisotope or fluorescer and the test substance to the plate. Next, with the amount of labeling substance bound onto the plate as an indicator, the binding of parasporin-1 to beclin-1 polypeptide in the presence of the test substance is measured, and compared with the binding in the absence of the test substance.

Alternatively, parasporin-1 and beclin-1 polypeptide are brought into contact with each other in the presence of a test substance by adding a solution containing parasporin-1 labeled with an appropriate fluorescer and the test substance to a cell expressing beclin-1 polypeptide on the cell surface. Next, the binding of parasporin-1 to beclin-1 polypeptide on the cell surface in the presence of the test substance is measured by flow cytometry, and compared with the binding in the absence of the test substance.

Then, on the basis of the comparative results for binding and dissociation rates or the amount bound, a test substance that inhibits the binding of parasporin-1 and beclin-1 polypeptide is selected as a substance capable of inhibiting the cytotoxicity of parasporin-1.

Because a substance which can be obtained by the screening method (I) of the present invention is capable of inhibiting the manifestation of cytotoxicity by parasporin-1, it is useful as a candidate substance for the prevention/treatment of cell death and poisoning that can be induced by parasporin-1. A substance which can be obtained by the screening method (I) of the present invention is also useful in analyzing the mechanism for the manifestation of cytotoxicity by parasporin-1.

### 4. Combination Comprising Parasporin-1 and Beclin-1 Polypeptide

The present invention provides a combination comprising parasporin-1 and beclin-1 polypeptide (the combination of the present invention). The combination of the present invention is useful as a reagent for screening for a substance capable of inhibiting the cytotoxicity of parasporin-1.

In an embodiment, parasporin-1 and beclin-1 polypeptide are comprised in the combination of the present invention while in a separated state. In this embodiment, parasporin-1 and beclin-1 are housed in different containers, respectively, and the plurality of containers are housed in one package.

In another embodiment, parasporin-1 and beclin-1 polypeptide are comprised in the combination of the present invention while in a mixed state, to constitute a composition.

The parasporin-1 comprised in the combination of the present invention is the same as one that can be used in the screening method (I) of the present invention.

The beclin-1 polypeptide comprised in the combination of the present invention is the same as one that can be used in the screening method (I) of the present invention. The beclin-1 polypeptide comprised in the combination of the present invention is preferably an isolated and purified polypeptide.

Using the combination of the present invention, it is possible to easily perform the screening method (I) of the present invention.

5. Agent and Method for Inducing Cell Death Using an Antibody that Specifically Recognizes Beclin-1 Polypeptide

As stated in an Example described below, when an effective amount of an antibody that specifically recognizes beclin-1 polypeptide is administered, the antibody binds to beclin-1 on the cell surface of a target cell, resulting in the induction of cell death (particularly apoptosis) to the cell as with the use of parasporin-1. Therefore, by administering an effective amount of beclin-1 polypeptide or an antibody that specifically recognizes beclin-1 polypeptide, the binding of parasporin-1 to beclin-1 polypeptide on the cell surface is inhibited, whereby the cytotoxicity of parasporin-1 is inhibited.

A targeted cell for the agent (II) of the present invention is a cell expressing beclin-1 polypeptide on the cell surface. Targeted cells for the agent (II) of the present invention include cultured cells, cells separated from living organisms, cells comprised in tissues separated from living organisms, cells in living organisms and the like. The cell is a tumor cell or non-tumor cell, preferably a tumor cell.

As an antibody that specifically recognizes beclin-1 polypeptide, that can be used in the agent (II) of the present invention, the same as one that can be used in the above-described agent (I) of the present invention can be mentioned. The antibody is preferably an antibody that exhibits an activity to induce cell death to a beclin-1-expressing cell when coming into contact with the cell. The epitope recognized by the antibody used in the agent (II) of the present invention is not particularly limited, as far as the antibody possesses an activity to induce cell death. Because a polyclonal antibody capable of recognizing whole beclin-1 polypeptide is potently active to induce cell death, it is preferably used in the agent (II) of the present invention. Such a polyclonal antibody can be acquired by administering a polypeptide comprising the full-length amino acid sequence of beclin-1 polypeptide, along with a commercially available adjuvant (for example, Freund complete or incomplete adjuvant), to an animal subcutaneously or intraperitoneally about 2 to 4 times at intervals of 2 to 3 weeks (the antibody titer of partially drawn serum should be measured by a publicly known antigen-antibody reaction, and an elevation thereof should be confirmed in advance), collecting whole blood about 3 to 10 days after final immunization, and purifying the antiserum. Animals to which the antigen is to be administered include non-human mammals such as rats, mice, rabbits, goat, guinea pigs, and hamsters.

The agent (II) of the present invention may contain, in addition to the above-described antibody that specifically recognizes beclin-1 polypeptide, an optionally chosen carrier, for example, a pharmaceutically acceptable carrier.

As examples of the pharmaceutically acceptable carrier, excipients such as sucrose and starch; binders such as cellulose and methylcellulose; disintegrants such as starch and carboxymethylcellulose; lubricants such as magnesium stearate and Aerosil; flavoring agents such as citric acid and menthol; preservatives such as sodium benzoate and sodium hydrogen sulfite; stabilizers such as citric acid and sodium citrate; suspending agents such as methylcellulose and polyvinylpyrrolidone; dispersing agents such as surfactants; diluents such as water and physiological saline; base waxes; and the like can be mentioned, which, however, are not to be construed as limiting.

The content of an antibody that specifically recognizes beclin-1 polypeptide in the agent (II) of the present invention is, for example, about 0.01 to 100% by weight of the entire pharmaceutical composition.

In an embodiment, the agent (II) of the present invention is used in vivo. In this case, the inhibitor is orally or parenterally administered to a subject animal. As preparations suitable for oral administration, liquids, capsules, sachets, tablets, suspensions, emulsions and the like can be mentioned. Preparations suitable for parenteral administration (for example, subcutaneous injection, intramuscular injection, topical injection, intraperitoneal administration and the like) include aqueous and non-aqueous isotonic sterile injectable liquids, which may contain an antioxidant, a buffer solution, a bacteriostatic agent, an isotonizing agent and the like. Aqueous and non-aqueous sterile suspensions can also be mentioned, which may contain a suspending agent, a solubilizer, a thickening agent, a stabilizer, an antiseptic and the like. These preparations can be encapsulated in containers such as ampoules and vials for unit dosage or a plurality of dosages. It is also possible to freeze-dry the active ingredient and a pharmaceutically acceptable carrier, and store the preparation in a state that may be dissolved or suspended in an appropriate sterile vehicle just before use.

When the agent (II) of the present invention is used in vivo, the dosage of the agent (II) of the present invention varies depending on the activity and choice of the active ingredient, seriousness of illness, recipient animal species, the recipient's drug receptivity, body weight, age, and the like, and cannot be generalized; however, the dosage is normally about 0.001 to about 500 mg/kg, based on the amount of active ingredient, per day for an adult human.

The agent (II) of the present invention is safely administered to the above-described mammal in a way such that the active ingredient thereof will be delivered to a targeted cell (a cell expressing beclin-1 on the cell surface).

In another embodiment, the agent (II) of the present invention is used in vitro. In this case, an antibody that specifically recognizes beclin-1 polypeptide is administered (added) to a medium containing a targeted cell (a cell that expresses beclin-1 on the cell surface).

When the agent (II) of the present invention is used in vitro, the dosage of the agent (II) of the present invention varies depending on the activity and choice of the active ingredient and the like, and cannot be generalized; however, the dosage is normally about 0.1 to 500 μg/ml, based on the final concentration of active ingredient in the culture.

The agent (II) of the present invention is useful as an antitumor agent in preventing and treating a tumor (preferably a tumor expressing beclin-1 polypeptide on the cell surface). The tumor is exemplified by, but not limited to, cervical carcinoma, gastric cancer, lung cancer, colorectal cancer, brain tumor, ovarian cancer, prostatic cancer, liver cancer, malignant lymphoma and the like.

### 6. Screening Method for Substances Capable of Inducing Cell Death

As stated above, an antibody that specifically recognizes beclin-1 polypeptide or parasporin-1 binds to beclin-1 polypeptide expressed on the cell surface to induce cell death (apoptosis) to the cell. Therefore, any substance, like parasporin-1, capable of binding to beclin-1 polypeptide is capable of inducing cell death to cells that express beclin-1 on the cell surface. Therefore, the present invention provides a method of screening for a substance capable of inducing cell death (apoptosis), comprising the steps shown below (the screening method (II) of the present invention):
(I) evaluating the degree of binding of a test substance to beclin-1 polypeptide, and
(II) selecting a test substance that has bound to beclin-1 polypeptide as a substance capable of inducing cell death.

The step (I) can further comprise comparing the degree of binding of the test substance to beclin-1 polypeptide with the degree of binding of the test substance to a polypeptide other than beclin-1 (control polypeptide).

The test substance subjected to the screening method (II) of the present invention, as in the screening method (I), may be any commonly known compound or a novel compound.

In the screening method (II) of the present invention, the same beclin-1 polypeptide as one used in the screening method (I) can be used. In the screening method (II) of the present invention, as in the screening method (I), an isolated and purified beclin-1 polypeptide or beclin-1 polypeptide expressed on the cell surface of a cell naturally or forcibly expressing beclin-1 is used as beclin-1 polypeptide.

The evaluation in the step (I) is normally performed in an appropriate culture medium or buffer solution. The degree of binding of a test substance to beclin-1 polypeptide can be evaluated, as in the screening method (I), by a method of interaction analysis well known in the art, for example, surface plasmon resonance, binding assay, immunological techniques and the like.

For example, a test substance and beclin-1 polypeptide are brought into contact with each other by immobilizing beclin-1 polypeptide on a chip, and loading a solution containing the test substance onto the chip. Next, the binding and dissociation of the test substance to or from beclin-1 polypeptide are measured by the surface plasmon resonance method, and compared with the binding and dissociation of the test substance to or from the control peptide when the control peptide is immobilized on the chip.

Alternatively, a test substance and beclin-1 polypeptide are brought into contact with each other by immobilizing beclin-1 polypeptide on a plate, and adding a solution containing the test substance labeled with an appropriate radioisotope or fluorescer to the plate. Next, with the amount of labeling substance bound onto the plate as an indicator, the binding of the test substance to beclin-1 polypeptide is measured, and compared with the binding to the control polypeptide.

Alternatively, a test substance and beclin-1 polypeptide are brought into contact with each other by adding a solution containing the test substance labeled with an appropriate fluorescer to a cell expressing beclin-1 polypeptide on the cell surface. Next, the binding of the test substance to beclin-1 polypeptide on the cell surface is measured by flow cytometry, and compared with the binding of the test substance to a control cell not expressing beclin-1.

Then, on the basis of the comparative results for binding and dissociation rates or the amount bound, a test substance that has bound to beclin-1 polypeptide is selected as a substance (candidate substance) capable of inducing cell death.

The screening method (II) of the present invention may comprise after the step (II) a step for bringing a substance selected through the step (II) into contact with a cell expressing beclin-1 on the cell surface, and selecting a substance that has induced cell death (preferably apoptosis) to the cell as a substance confirmed to induce cell death.

Contact of the test substance and the cell expressing beclin-1 on the cell surface is performed in a culture medium. A culture medium is chosen as appropriate according to the kind of cell; examples include a minimal essential medium (MEM), Dulbecco's Modified Eagle's Medium (DMEM) and the like containing about 5 to 20% fetal calf serum. Culturing conditions are also determined as appropriate; for example, the pH of the medium is about 6 to about 8, culturing temperature is normally about 30 to about 40° C., and culturing time is about 12 to about 72 hours. Cell death induction can be evaluated by the TUNNEL method, DNA ladder detection and the like.

Substances obtained by the screening method (II) of the present invention are useful as candidate substances for the development of new antitumor agents.

The present invention is explained in more detail in the following by referring to Examples and the like, which are not to be construed as limitative.

### Examples

### 1. Binding of Parasporin-1 to HeLa Cell

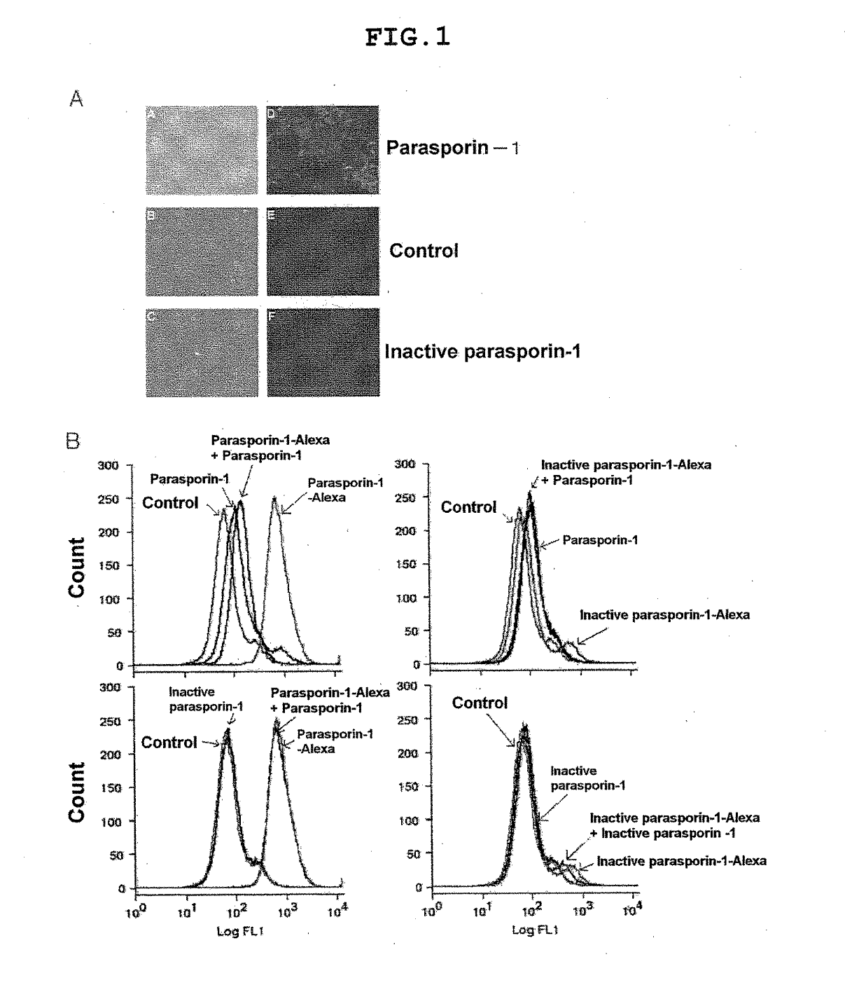

To confirm that parasporin-1 can bind to the target cell surface, HeLa cells were treated with parasporin-1 labeled with Alexa Fluor 488 dye, and immunostained with an anti-Alexa Fluor 488 dye antibody. The cells treated with active parasporin-1 could be stained with a fluorescence antibody, but staining could not be detected in control and the cells treated with inactive parasporin-1 treated at 100° C. for 5. min (FIG. 1A). HeLa cells were treated with parasporin-1 labeled with Alexa Fluor 488 dye, and the binding of parasporin-1 to the cells was analyzed by flow cytometry (FIG. 1B). The cells treated with active labeled parasporin-1 showed increased fluorescence, and the increased fluorescence disappeared by adding non-labeled parasporin-1 in much excess (40-fold of labeled parasporin-1) (FIG. 1B, upper left panel).

2. Comparison of Binding to Cell Lines with Various Parasporin-1 Sensitivity

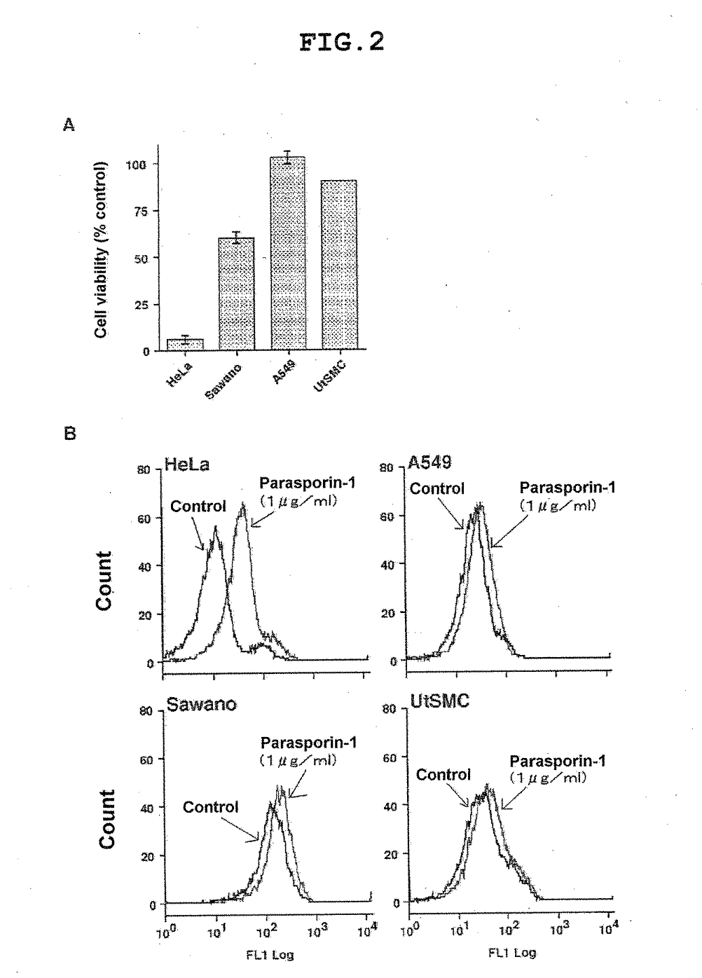

Parasporin-1 is a cytotoxin with high cell specificity. FIG. 2A shows sensitivity of HeLa, A549, Sawano and normal uterus smooth muscle cells (UtSMC) to parasporin-1 (1 μg/ml). To show correlation between parasporin-i sensitivity of cell and binding of parasporin-1 to cell membrane, the binding property of parasporin-1 to various cells was analyzed by flow cytometry. HeLa cell showed parasporin-1 dependent peak shift but other cell lines hardly showed a peak shift.

### 3. Screening and Identification of Parasporin-1 Receptor Using Photoreactive Chemical Crosslinking Agent

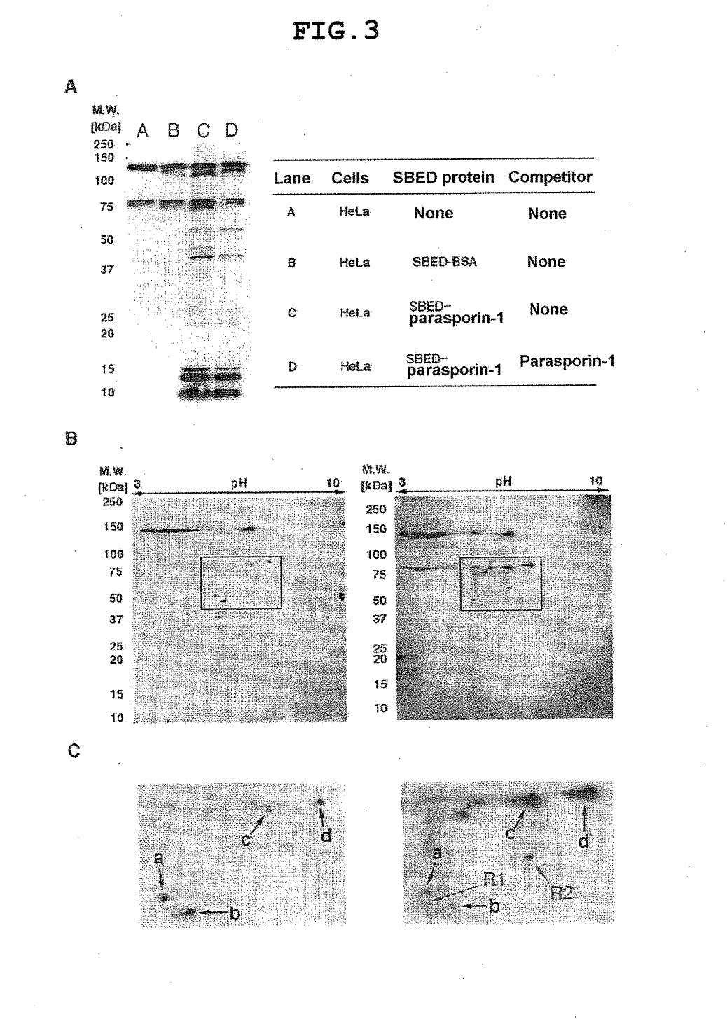

Parasporin-1 receptor was screened for using parasporin-1 labeled with a photoreactive chemical crosslinking agent (SEED). As a result of chemical crosslinking, since biotin transfer occurs in a protein that has interacted with parasporin-1, a protein of HeLa cell to which biotin was transferred was screened for by Western blotting. As a control, SBED-labeled BSA was used. As a result of Western blot, proteins to which biotin was transferred in a parasporin-1 dependent manner were observed (FIG. 3A, lane C). It was further confirmed that biotin translocation clearly disappeared by the coexistence of non-labeled parasporin-1 in a part of proteins in which biotin transfer had been observed (FIG. 3A, lane D). Although the protein was separated by one-dimensional electrophoresis and identification of a protein showing biotin translocation was tried, the identification failed since separation of protein was insufficient. Thus, the protein of the cell was further separated by two-dimensional electrophoresis, and a protein showing induction of biotin translocation was screened for. HeLa cells were treated with SBED-labeled BSA or SBED-labeled parasporin-1, and a crosslinking reaction was performed. The cell membrane was recovered from the treated cells, and a protein was separated by two-dimensional electrophoresis. For detection of a Biotin-transferred protein, Western blotting using avidin-HRP was performed (FIG. 3B). As a result of two-dimensional electrophoresis, protein spots (R1, R2) were observed in pH 6 to pH 8, molecular weight 37-80 kDa, where parasporin-1 dependent biotin translocation was induced (FIG. 3C, right panel). R1 and R2 were prepared by two-dimensional electrophoresis. The prepared protein was identified by mass finger printing method. As a result, R1 and R2 were tropomodulin 3 (TMOD-3) and beclin-1, respectively.

### 4. Inhibitory Effect of Antibody on Cell Binding of Parasporin-1

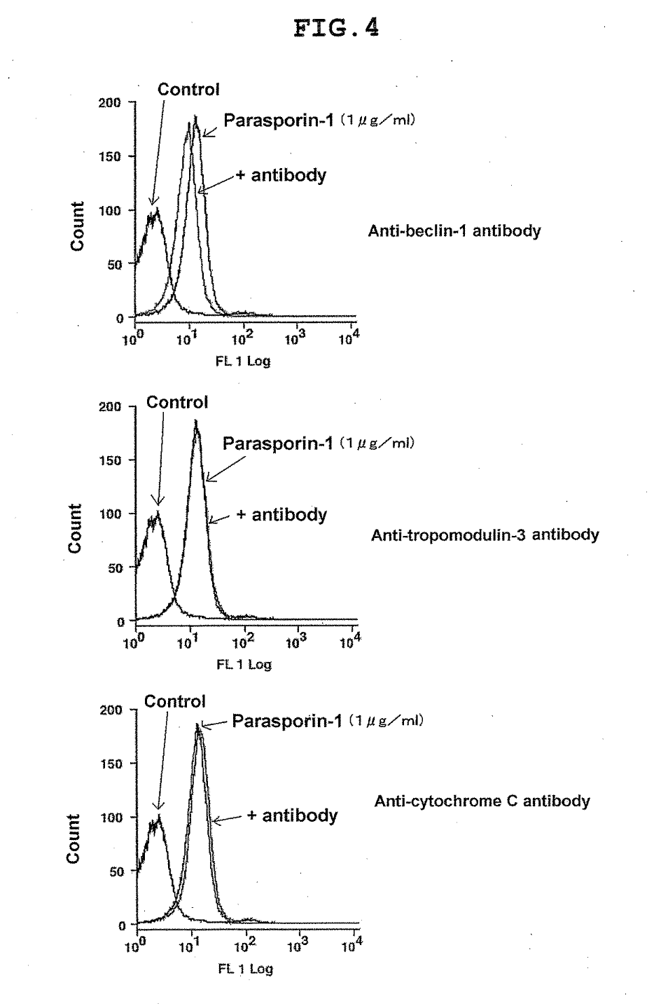

To confirm that parasporin-1 receptor candidate protein identified by mass finger printing method has a parasporin-1 receptor function, inhibition of cell membrane binding of parasporin-1 by antibodies against each candidate protein was examined. HeLa cells were treated in advance with anti-beclin-1 antibody, anti-cytochrome C antibody or anti-TMOD-3 antibody, and binding of fluorescence-labeled parasporin-1 to HeLa cells was detected by flow cytometry. As anti-beclin-1 antibody, a polyclonal antibody manufactured by Cell Signaling Technology was used. The polyclonal antibody was prepared by immunizing a rabbit with a synthesized partial peptide (KLH-conjugated) corresponding to the residue adjacent to threonine 72 of human beclin-1. The binding of parasporin-1 to HeLa cell was inhibited by anti-beclin-1 antibody (FIG. 4A), but it was not observed in anti-TMOD-3 antibody and anti-cytochrome C antibody (FIGS. 4B and C). In addition, anti-beclin-1 antibody that inhibits binding of parasporin-1 to HeLa cell could also partially inhibit cytotoxicity of parasporin-1 against HeLa cell.

### 5. Localization of Beclin-1 in Cell Membrane

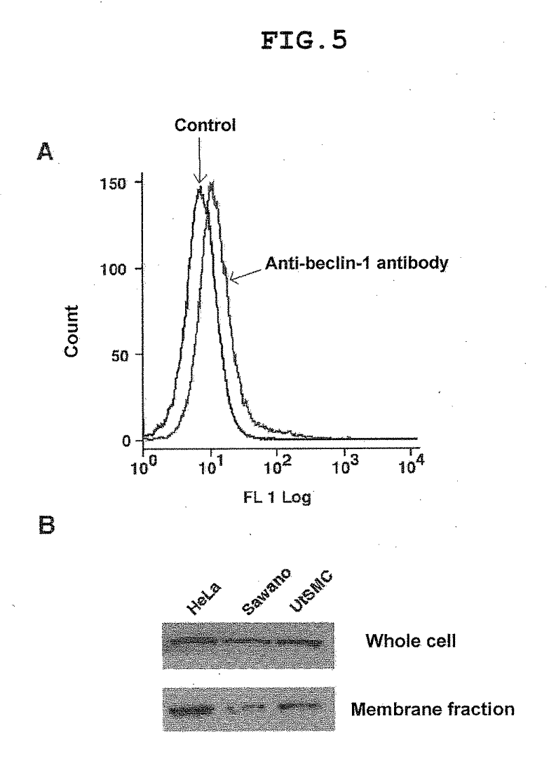

To show that beclin-1 is present on the cell membrane, HeLa cells were treated with anti-beclin-1 antibody (manufactured by Cell Signaling Technology), and the anti-beclin-1 antibody on the HeLa cell surface was detected with a fluorescence antibody by flow cytometry. As a result, fluorescence peak shift was observed in an anti-beclin-1 antibody dependent manner, suggesting that beclin-1 is present on the cell surface of HeLa cells (FIG. 5A). Then, the cellular components were fractionated by ultracentrifugation, and whether the membrane fractions including organelle contain beclin-1 was examined. Beclin-1 was detected by Western blotting. The experiment was performed not only with HeLa cells but also with other cell lines (Sawano, UtSMC) with different parasporin-1 sensitivity. The content of beclin-1 present in the cells was not much different between the cell lines (FIG. 5B, upper panel). However, the content of beclin-1 present in membrane fractions in Sawano and UtSMC was low as compared to HeLa cells (FIG. 5B, lower panel).

### 6. Cytotoxicity of Anti-Beclin-1 Antibody

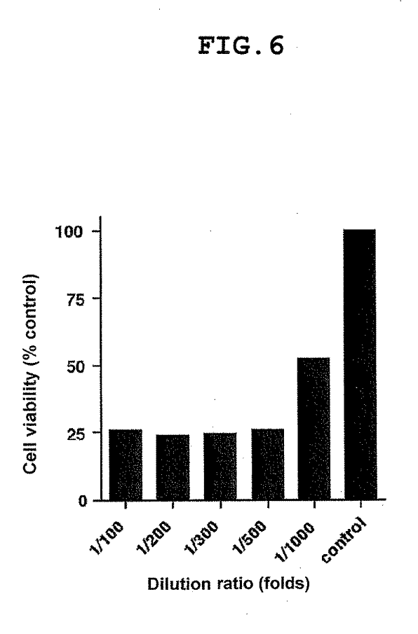

Cell death is induced by binding of parasporin-1 to beclin-1. Therefore, whether anti-beclin-1 antibody having higher affinity than parasporin-1 shows a cytotoxicity induction effect similar to that of parasporin-1 was examined. The anti-beclin-1 antibody used for this experiment was a polyclonal antibody manufactured by Affinity Bioreagents. The polyclonal antibody was prepared by immunizing a rabbit with a human beclin-1 protein (whole protein). The anti-beclin-1 antibody was diluted and HeLa cells were treated with the antibody at 37° C. for 20 h. The survival rate of the cell was calculated by a color development method using an MTT reagent. The cell survival rate did not decrease in the control (pre-immunized rabbit serum), whereas the cell survival rate remarkably decreased with the anti-beclin-1 antibody (FIG. 6). However, cell death was scarcely induced by the above-mentioned anti-peptide antibody (manufactured by Cell Signaling Technology) against beclin-1 having a limited binding region.

### Discussion

Experimental results suggesting the presence of a proteinous receptor of parasporin-1 on the HeLa cell membrane have been obtained heretofore. In this experiment, the receptor of cytotoxin parasporin-1 was identified to be beclin-1 by using a photoreactive chemical crosslinking agent.

The binding ability of parasporin-1 to the cell membrane surface of cell lines having various sensitivities to parasporin-1 showed high correlation with parasporin-1 sensitivity. Therefrom it was assumed that parasporin-1 sensitivity of a cell line is determined by the binding of parasporin-1 to the cell surface, namely, the amount or the presence or absence of a receptor. The amount of beclin-1 in the membrane fraction containing organelle of highly sensitive HeLa cell was high and that in the cells (Sawano and UtSMC) having low sensitivity was small. However, the content of beclin-1 in the whole cells of HeLa, Sawano and UtSMC was almost the same. These results suggest that the selectivity of cells to parasporin-1 toxicity depends on expression of beclin-1 on the cell surface. In addition, it is shown that a parasporin-1 treatment induces an increase in the intracellular calcium concentration of parasporin-1 sensitive cells, which triggers cytotoxicity (Katayama, H. et al., J. Biol. Chem. 282, 7742-7752 (2007)). This suggests that beclin-1 that functions as a extracellularly exposed receptor of parasporin-1 regulates the mechanism of calcium influx.

Beclin-1 has been identified as a protein relating to autophagy, and acts as a cancer suppressive factor. In addition, beclin-1 has been reported to be an intracellular protein that interacts with Bcl-2 and Bcl-X_{L}, which are the factors regulating apoptosis. However, it is not known that beclin-1 appears on the cell surface, and functions as a receptor. Therefore, it can be said that parasporin-1 has indicated new and unknown function of beclin-1. While the physiological meaning of beclin-1 present on the cell surface is completely unknown at the present stage, beclin-1 is assumed to be at least involved in the cellular phenomena other than autophagy and apoptosis induction.

Parasporin-1 is a toxic protein showing selective toxicity to the cell, and localization of its receptor, beclin-1, on the cell membrane surface was found in the parasporin-1 sensitive cells. Moreover, a treatment of HeLa cells (parasporin-1 high sensitive cell) with an anti-beclin-1 antibody resulted in the apoptosis induction via caspase-8, like that of parasporin-1. The foregoing suggests that cell membrane localization of beclin-1, which is a parasporin-1 receptor, can be used as one marker for distinguishing the kind of cells, and that the development of a new antitumor agent targeting extracellularly exposed beclin-1 is possible.

### INDUSTRIAL APPLICABILITY

The present invention provides various uses of beclin-1 as a receptor of parasporin-1.

Using the method of the present invention for determining sensitivity to parasporin-1, it is possible to objectively determine the sensitivity of various cells to parasporin-1 on the basis of the expression level of parasporin-1 receptor definitely supported by molecular biologically.

Using the inhibitor of the cytotoxicity of parasporin-1 of the present invention, it is possible to potently suppress the cell death (apoptosis) induced by parasporin-1.

Using the cell death inducer of the present invention, it is possible to induce cell death (apoptosis) specifically to cells expressing beclin-1, and the inducer is useful as a cell-specific antitumor agent.

This application is based on a Japanese patent application No. 2008-090190 (filing date: Mar. 31, 2008, the contents of which are incorporated in full herein.

1. A method of determining the sensitivity of a cell to parasporin-1, comprising measuring the expression level of beclin-1 polypeptide on the cell membrane or in a membrane fraction of a cell, and determining the sensitivity of the cell to parasporin-1 on the basis of a positive correlation between the expression level of beclin-1 polypeptide and the sensitivity to parasporin-1.

2. The method according to claim 1, wherein the measurement of the expression level of beclin-1 polypeptide is performed using an antibody that specifically recognizes beclin-1 polypeptide.

3.-4. (canceled)

5. A method of inhibiting the cytotoxicity of parasporin-1, comprising administering beclin-1 polypeptide or an antibody that specifically recognizes beclin-1 polypeptide and is capable of inhibiting the specific binding of beclin-1 polypeptide and parasporin-1 polypeptide.

6. A method of screening for a substance capable of inhibiting the cytotoxicity of parasporin-1, comprising the steps shown below:
(I) evaluating the degree of binding of parasporin-1 to beclin-1 polypeptide in the presence of a test substance, and
(II) selecting a substance that has inhibited the binding of parasporin-1 to beclin-1 polypeptide as a substance capable of inhibiting the cytotoxicity of parasporin-1.

7. A combination comprising parasporin-1 and beclin-1 polypeptide.

8.-9. (canceled)

10. A method of inducing cell death, comprising bringing an antibody that specifically recognizes beclin-1 polypeptide into contact with a cell expressing beclin-1 polypeptide on the cell surface, wherein the antibody exhibits an activity to induce cell death to a cell expressing beclin-1 polypeptide on the cell surface when coming into contact with the cell.

11. The method according to claim 10, wherein the cell is a tumor cell.

12. A method of screening for a substance capable of inducing cell death, comprising the steps shown below:
(I) evaluating the degree of binding of a test substance to beclin-1 polypeptide, and
(II) selecting a test substance that has bound to beclin-1 polypeptide as a substance capable of inducing cell death.

13. The method according to claim 12, wherein the beclin-1 polypeptide used in (I) is beclin-1 polypeptide expressed on the cell membrane.

14.-15. (canceled)

16. A method of inducing cell death of a cell expressing beclin-1 polypeptide on the cell surface in a mammal, comprising administering to the mammal an effective amount of an antibody that specifically recognizes beclin-1 polypeptide and exhibits an activity to induce cell death to a cell expressing beclin-1 polypeptide on the cell surface when coming into contact with the cell.

17. A method of preventing or treating a tumor expressing beclin-1 polypeptide on the cell surface in a mammal, comprising administering to the mammal an effective amount of an antibody that specifically recognizes beclin-1 polypeptide and exhibits an activity to induce cell death to a cell expressing beclin-1 polypeptide on the cell surface when coming into contact with cell.


SEQUENCE LISTING (6 sequences)

SEQ ID NO: 1 - DNA, 2144 bp, Homo sapiens
  CDS (134)..(1486)
gctgaagacagagcgatggtagttctggaggcctcgctccggggccgacccgaggccacagtgcctccgcggtagaccggacttgggtgacgggctccgggctcccgaggtgaagagcatcgggggctgagggatggaagggtctaagacgtccaacaacagcaccatgcaggtgagcttcgtgtgccagcgctgcagccagcccctgaaactggacacgagtttcaagatcctggaccgtgtcaccatccaggaactcacagctccattacttaccacagcccaggcgaaaccaggagagacccaggaggaagagactaactcaggagaggagccatttattgaaactcctcgccaggatggtgtctctcgcagattcatccccccagccaggatgatgtccacagaaagtgccaacagcttcactctgattggggaggcatctgatggcggcaccatggagaacctcagccgaagactgaaggtcactggggacctttttgacatcatgtcgggccagacagatgtggatcacccactctgtgaggaatgcacagatactcttttagaccagctggacactcagctcaacgtcactgaaaatgagtgtcagaactacaaacgctgtttggagatcttagagcaaatgaatgaggatgacagtgaacagttacagatggagctaaaggagctggcactagaggaggagaggctgatccaggagctggaagacgtggaaaagaaccgcaagatagtggcagaaaatctcgagaaggtccaggctgaggctgagagactggatcaggaggaagctcagtatcagagagaatacagtgaatttaaacgacagcagctggagctggatgatgagctgaagagtgttgaaaaccagatgcgttatgcccagacgcagctggataagctgaagaaaaccaacgtctttaatgcaaccttccacatctggcacagtggacagtttggcacaatcaataacttcaggctgggtcgcctgcccagtgttcccgtggaatggaatgagattaatgctgcttggggccagactgtgttgctgctccatgctctggccaataagatgggtctgaaatttcagagataccgacttgttccttacggaaaccattcatatctggagtctctgacagacaaatctaaggagctgccgttatactgttctggggggttgcggtttttctgggacaacaagtttgaccatgcaatggtggctttcctggactgtgtgcagcagttcaaagaagaggttgagaaaggcgagacacgtttttgtcttccctacaggatggatgtggagaaaggcaagattgaagacacaggaggcagtggcggctcctattccatcaaaacccagtttaactctgaggagcagtggacaaaagctctcaagttcatgctgacgaatcttaagtggggtcttgcttgggtgtcctcacaattttataacaaatgacttttttccttagggggaggtttgccttaaaggcttttaattttgttttgtttgcaaacatgttttaaattaaattcgggtaatattaaacagtacatgtttacaataccaaaaaagaaaaaatccacaaaagccactttattttaaaatatcatgtgacagatactttccagagctacaacatgccatctatagttgccagccctggtcagttttgattcttaaccccatggactcctttccctttcttctctgaaaaaaactaatttaaatttgcttttcttttttttaactgagttgaattgagattgatgtgttttcactggatttttatctctctcaacttcctgcacttaacaatatgaaatagaaacttttgtctttactgagatgaggatatgtttgagatgcacagttggataatgtgggaaaatgacatctaagctttacctggtcaccatgtgatgtgatcagatgcttgaaatttaacacttttcacttggttcttatactgaatgccgactctgctctgtgttagagatatgaaatggtgtttgatactgtttgagacattatggagagatttaattatttgtaataaaagatttgctgcagtctgaaaactgaaaaaaaaaaaaaaaaaaaaaaaaaaaaaaaa

SEQ ID NO: 2 - protein, 450 aa, Homo sapiens
MEGSKTSNNSTMQVSFVCQRCSQPLKLDTSFKILDRVTIQELTAPLLTTAQAKPGETQEEETNSGEEPFIETPRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQMELKELALEEERLIQELEDVEKNRKIVAENLEKVQAEAERLDQEEAQYQREYSEFKRQQLELDDELKSVENQMRYAQTQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 3 - DNA, 2031 bp, Mus musculus
  CDS (139)..(1485)
agtcttcagctagccgaccgggaagtagctgaagaccgggcgatgggaactctggaggtctcgctccggggcggacccgaggccaccgtgcccccgcggtagaacgagcctgggtggcgggctccgggctcctgaggcatggaggggtctaaggcgtccagcagcaccatgcaggtgagcttcgtgtgccagcgctgtagccagcctctgaaactggacacgagcttcaagatcctggaccgggtcaccatccaggaactcacagctccattacttaccacagcccaggcgaaaccaggagagacccaggaggaagaggctaactcaggagaggagccatttattgaaactcgccaggatggtgtctctcgaagattcatccccccagccaggatgatgtctacagaaagtgctaatagcttcactctgatcggggaggcatctgatggtggcaccatggagaacctcagccggagactcaaggtcactggagacctgtttgacatcatgtcgggccagaccgatgtggatcacccgctgtgtgaggaatgcacagacactcttttagaccagctggacactcagctcaatgtcactgagaatgaatgtcagaactacaaacgctgtttggagatcctagagcagatgaatgaggatgacagtgagcagctgcagagggagctgaaggagctggccttggaggaggagaggctgatccaggagctggaagatgtggaaaagaaccgcaaggtggtggcagagaacctggagaaggtccaggctgaggcggagagattggaccaggaggaagctcagtaccagcgggagtatagtgagtttaaaaggcagcagctggagttggatgacgaactcaagagtgtggagaaccaggtgcgctacgcccagatccagctggacaagctcaagaaaaccaatgtcttcaatgccaccttccacatctggcacagcggacagtttggcacaatcaataatttcagactgggtcgcttgcccagtgttcctgtggagtggaatgaaatcaatgctgcctggggccagacagtgctgctgctccatgctttggccaataagatgggtctgaagtttcagaggtaccgacttgttccctatggaaatcattcctatctggagtctctgacagacaaatctaaggagttgccgttatactgttctgggggtttgcggtttttctgggacaacaagtttgaccatgcaatggtagcttttctggactgtgtgcagcagttcaaagaagaggtggaaaaaggagagactcgattttgtcttccgtacaggatggacgtggagaaaggcaagattgaagacactggaggcagtggcggctcctattccatcaaaacccagtttaactcggaggagcagtggacaaaagcgctcaagttcatgctgaccaatctcaagtggggtcttgcctgggtgtcctcacagttctataacaagtgacttgctccttaggggatgtttgcctttaaggttttatactttgtttggtttggaaagatgctttaaattaaatttgggtaatattaaaccacatgtttacaataccaaaatccacaaaagctactttattttcaaatatgacagatagtttccagagtacgccatgtatagcaaagaaccctgccatagttttgactcagccccatgcatcctttccctctttcctgaaaacaactaatttaaatttgctttgttttcttttttaagttgaattgacgttaatgtgttttcactggattttatctctctcaacttcctgcacttaaaatttgaaacagcaaaggtttgagatgagatgcttgtggcacacagttgggtgatgtggggaaaggacaccgggtcaggagttgcaagtttaactccgtcctcacttgtagcattgaatgcctcctgtgctgtctagtgggactacagaatgctgtttgatactgtgtgcgacgtggagagatttaattatttgtaataaaggatttgctatggtctatt

SEQ ID NO: 4 - protein, 448 aa, Mus musculus
MEGSKASSSTMQVSFVCQRCSQPLKLDTSFKILDRVTIQELTAPLLTTAQAKPGETQEEEANSGEEPFIETRQDGVSRRFIPPARMMSTESANSFTLIGEASDGGTMENLSRRLKVTGDLFDIMSGQTDVDHPLCEECTDTLLDQLDTQLNVTENECQNYKRCLEILEQMNEDDSEQLQRELKELALEEERLIQELEDVEKNRKVVAENLEKVQAEAERLDQEEAQYQREYSEFKRQQLELDDELKSVENQVRYAQIQLDKLKKTNVFNATFHIWHSGQFGTINNFRLGRLPSVPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 5 - DNA, 2140 bp, Rattus norvegicus
  CDS (225)..(1571)
tggatctggaccaggggtccttgcgtcagctctcgtcaaggcgtcacttctgggcccgggcggaagtcttcagctagccgaccgggaagtagctgaagaccgggcgatgggaactctggaggtctcgctctggggcggacccgaggccaccgttcccccgcggtagagcgagcctaggtggcgggctccggggtcctgaggtgaagagcgtcggggcctaaagaatggaggggtctaaggcgtccagcagcaccatgcaggtgagcttcgtgtgccagcgctgtagccagcctctgaaactggacacgagcttcaagatcctggaccgagtgaccattcaggaactcacagctccattacttaccacagcccaggcgaaaccaggagagagccaggaggaagaggctaactcaggagaggagccatttattgaaactcgccaggatggtgtctctcgaagattcatccccccagccaggatgatgtctacagaaagtgctaatagcttcactctgatcggggaggcatctgatggtggcaccatggagaacctcagccggagactcaaggtcactggagacctttttgacatcatgtctggccagacagatgtggatcacccactgtgtgaggaatgcacagatactcttttagaccagctggacactcagctcaatgttactgaaaacgagtgtcagaactacaaacgctgtttggagatgttggagcaaatgaatgagggcgacagtgaacagctacagagggagctgaaggagttggccttggaggaggagaggctgatccaggagctggaagatgtggaaaaaaaccgaaaggtggtggcagaaaacctggagaaggtccaggctgaggcggagagactggaccaggaggaagctcagtaccagcgagaatatagtgaatttaaaaggcagcagctggagctggatgatgagctcaagagtgtagagaaccagatgcgctatgcccagatgcagctggacaagctcaagaaaaccaatgtcttcaatgcgaccttccatatctggcacagcggacaatttggcacgatcaataatttcagactgggtcgcttgcccagtgctcctgtggaatggaatgaaatcaatgctgcctggggccagacagtgttgttgctccatgctttggccaataagatgggtctgaaatttcagaggtaccgacttgttccctatggaaatcactcgtatctggagtccctgacagacaaatctaaggagttgccgttgtactgttctgggggtttgcggtttttctgggacaacaagtttgaccatgcaatggtagcttttctggactgtgtgcagcagttcaaagaagaggtggaaaaaggagagactcgattttgtcttccgtacaggatggacgtggagaaaggcaagattgaagacactggaggcagtggcggctcctattccatcaaaacccagtttaactctgaggagcagtggacaaaggcgctcaagttcatgctgacgaatctcaagtggggtcttgcttgggtgtcctcacagttctataacaagtgacttgctccttaggggatatttgcctttaaggttttacattttgtttggtttggaaagatgctttaaattaaatttgggtaatattaaaccacatgtttacaataccagaatccacaaaagctactttattttacaatatgacagttttcagagcacagcacgccatgtatagcaaagagccctgccgtagtttggctcaaccccatgctgtcctttccctcttccctggaaacaactaatttaaatttgctttgttttcttttttaagttgaattgacattaatgtgttttcactggattttatctctctcaacttcctgtacttgaaacagaaaagttttgagatgagatgcttgtggcacacagttgggtaatgtggggaagggacaagaggtaaaggtttaactctgtcctcacttctagtattgaacgcttactgtgctaactattggaattacaaaatgctgtttgatactgtttgagatgtggaaggatttaattatttgtaataaaggatttgctatggtctattaattaaaaaaaaaaaaaaaaaaaaaaaaaaaaaaaaa

SEQ ID NO: 6 - protein, 448 aa, Rattus norvegicus
MEGSKASSSTMQVSFVCQRCSQPLKLDTSFKILDRVTIQELTAPLLTTAQAKPGESQEEEANSGEEPFIETRQDGVSRRFIPPARMMSTESANSFTLIGEASDGGTMENLSRRLKVTGDLFDIMSGQTDVDHPLCEECTDTLLDQLDTQLNVTENECQNYKRCLEMLEQMNEGDSEQLQRELKELALEEERLIQELEDVEKNRKVVAENLEKVQAEAERLDQEEAQYQREYSEFKRQQLELDDELKSVENQMRYAQMQLDKLKKTNVFNATFHIWHSGQFGTINNFRLGRLPSAPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK